(19) United States
(12) Patent Application Publication
(10) Pub. No.: US 2003/0224076 A1
(43) Pub. Date: Dec. 4, 2003

(54) Anti-inflammatory and chemopreventive composition

(76) Inventor: Bernd Haber, Mainz (DE)

Correspondence Address: ProPat, L.L.C., 2912 Crosby Road, Charlotte, NC, 28211-2815, US

(21) Appl. No.: 10/441,401

(22) Filed: May 20, 2003

(30) Foreign Application Priority Data
May 28, 2002 (DE) 102 23 856.1

(51) Int. Cl. A61K 35/78

(52) U.S. Cl. 424/757; 424/777

(57) ABSTRACT

The invention relates to an anti-inflammatory and chemopreventive composition which comprises carob products, especially carob fiber. The invention further relates to a process for producing these compositions and to the use thereof with the aim of reducing the risk, the prevention, the prophylaxis and the treatment of inflammatory disorders or cancers, especially of the gastrointestinal tract.

The invention relates to an anti-inflammatory and chemopreventive composition which comprises carob products, especially carob fiber. The invention further relates to a process for producing these compositions and to the use thereof with the aim of reducing the risk, the prevention, the prophylaxis and the treatment of inflammatory disorders or cancers, especially of the gastrointestinal tract.

### BACKGROUND OF THE INVENTION

An inflammation is a defence response by the body to internal or external stimuli (e.g. pressure, foreign bodies, radiation, microorganisms or pathological metabolic products). This may lead to reddening, heating, swelling, pain and functional impairments. The inflammatory response originates from the connective tissue of the blood vessels of the damaged organ or tissue. Through the mediation of hormone-like agents, various defence systems in the body are activated.

Inflammatory disorders of the gastrointestinal region encompass inter alia intestinal inflammations (enteritis), gastroenteritis, nonspecific irritable bowel syndrome and the chronic inflammatory bowel disorders ulcerative colitis and Crohn's disease.

Chronic inflammatory bowel disorders are manifested by symptoms such as irregular bowel movements, diarrhea, abdominal pain, nausea or loss of weight. With the chronic inflammatory bowel disorders, a distinction is normally made between ulcerative colitis and Crohn's disease, which entail different dietary and treatment measures. Whereas Crohn's disease may spread to all sections of the digestive tract and leaves behind discontinuous foci of inflammation, ulcerative colitis is confined to the large bowel region and extends almost continuously from the rectum. One problem is that dietary measures depend on the individual but also on the point in time within the particular progress of the disease. Causes suggested for these types of disorder are infectious factors (such as viruses, bacteria or fungi), genetic factors, dietary factors (e.g. sugar, protein), psychological factors and an overreaction of the immune defences. Chronic inflammatory disorders over many years greatly increase the risk of developing cancer.

Cancer is a multifactorial pathological state which is caused, but can also be influenced, by diverse factors. It is supposed that certain cancers can be avoided by correct diet and a healthy lifestyle. Types of cancer of the gastrointestinal tract such as cancer of the colon and stomach in particular are among the cancers which can be influenced most strongly by diet.

The start of the process of carcinogenesis is in most cases associated with contact of the DNA with carcinogenic substances. These may be produced endogenously or be introduced exogenously. Endogenous factors which may be involved are substances which are produced physiologically but which may damage DNA, and exogenous factors which may be mentioned are, besides viruses or radioactive emissions, in particular so-called procarcinogens which are genotoxic only after metabolic activation. Factors influencing the activity of detoxifying enzymes or DNA repair enzymes may also have a crucial influence on the development of cancer. Dietary constituents may intervene beneficially in the development of cancer in a wide variety of ways. Thus, for example, certain vitamins such as vitamin C or vitamin E or else certain plant constituents such as carotenoids or flavonoids show properties which may prevent possible damage by carcinogenic substances.

Colorectal carcinomas are among the most commonly occurring types of cancer of the gastrointestinal tract. The most important factors influencing these cancers are regarded as diet and physical activity. Inflammatory reactions are regarded as risk-promoting, whereas acetylsalicylic acid or other non-steroidal anti-inflammatory drugs have a protective effect. The proportion of colorectal carcinomas with a genetic cause is estimated as much less than 10%.

To date, inflammatory disorders of the gastrointestinal tract have been treated in particular with anti-inflammatory drugs such as corticosteroids, but also antibiotics and antidiarrheals. The corticosteroids are generally the most effective medicaments for treating acute episodes of chronic inflammatory bowel disorders, although they cannot be employed for a prolonged period. Long-term therapy with systemically active corticosteroids leads to side effects which frequently force a reduction in the dose or discontinuation of the therapy. These side effects include inter alia weight gain with truncal obesity, moon face, acne, adrenocortical atrophy, elevation of blood glucose and cholesterol, rise in blood pressure or osteoporosis.

For mild to moderate episodes of ulcerative colitis or to maintain freedom from symptoms, but also for specific types such as left-sided ulcerative colitis, 5-amino-salicylic acid is preferably used. With this too, possible side effects such as, for example, alterations in blood count or an impairment of renal function, but also nausea, vomiting, diarrhea and abdominal pain, are to be expected during long-term therapies.

Dietary measures are important in addition to medical therapy. They primarily attempt to contain and to counteract the undersupply of nutrients occurring due to the inflammatory disorders. Food constituents which may have a beneficial effect on the pathological state and which should be mentioned are viscous, efficiently swelling bulking agents such as, for example, pectin, oat bran, guar or ispaghula. In addition, polyunsaturated fatty acids appear to have an inhibitory effect on certain inflammatory diseases. Probiotics have also been suggested to have a beneficial effect on inflammatory bowel disorders. The food constituents have to date been mainly recommended as assisting particular dietary measures, with the diet best tolerated by the patient usually being found empirically.

Various protective dietary factors have been suggested in connection with cancers of the digestive tract. Besides reducing meat and meat products, fat and alcohol, a reduced risk of cancer of the gastrointestinal tract is associated with high fruit and vegetable consumption. Viscous bulking agents and resistant starch, which are good butyrate formers, but also dietary constituents such as vitamins C and E or else certain flavonoids are attributed with an important role as protective factors.

Food products have to date played a part in the treatment of cancers of the gastrointestinal tract (surgery, radiotherapy, chemotherapy) only in the form of a therapy-supporting and -supplementing diet. This aims particularly at adequately supplying the patient with nutrients and minerals, trace elements and vitamins.

As a food source which has been used but little to date, carob might become of interest in future. Carob is a fruit of the Mediterranean carob tree and has an exceptionally sweet taste owing to its high natural carbohydrate content. Carob itself is a good source of bulking agents and polyphenols. Water-soluble polyphenol-rich carob extracts show high antioxidant or else antimicrobial activity (Henis et al., Applied Microbiology, Vol. 12, No. 3, 1964, 204-209; Kumazawa et al., J. Agric. Food Chem. 2002, 50 373-377; Nutrinova, Caromax™ “New Perspectives”; 02/2002). It is also reported that products made from carob produced by the process described in U.S. Pat. Nos. 4,999,197 and 5,330,755 have an antidiarrheal effect (also U.S. Pat. No. 5,043,160). Roasted carob powder has been employed in various ways as supplementary dietary measure in the therapy of acute diarrhea, especially in infants (Loeb et al., J. Pediatr. Gastroenterol. Nutr., 8, 480-485, 1989). EP-A-0 616 780 describes a process for producing a carob product rich in bulking agents. The particular production process results in products which are rich in insoluble carob fibers and, besides their digestion-promoting effect, also show other additional health benefits such as lowering of cholesterol (Pérez-Olleros et al., J. Sci. Food Agric. 79, 173-178, 1999). There have been no reports on whether carob products, especially insoluble carob fibers, are able to have a beneficial effect on inflammatory reactions or the risk of cancer or development of cancer.

Previous approaches to reducing the risk of inflammatory disorders or cancers of the gastrointestinal tract are unsatisfactory. One of the basic problems, particularly in relation to protection from the disorders, is the elaborate change of diet based on dietary recommendations. Consumers' acceptance of this is low until they become really ill. For effective protection acceptable to the consumer it is therefore necessary to develop compact, highly effective and easily handled solutions, e.g. in the form of food products for daily consumption. The dietary factors which have to date been described as beneficial mainly have only a weakly supporting and often very nonspecific effect.

It would furthermore be desirable to reduce through suitable dietary measures the use and thus the frequently occurring side effects of medicaments, especially for inflammatory bowel disorders, but also cancers. There is thus the further need for food components which are able to reduce or improve particular risk factors of inflammatory disorders or cancers of the gastrointestinal tract and provide worthwhile support to drug therapy.

### BRIEF DESCRIPTIONS OF THE INVENTION

This is achieved by an anti-inflammatory and chemopreventive composition which comprises carob products, especially carob fibers.

### DETAILED DESCRIPTION OF THE INVENTION

Anti-inflammatory means for the purposes of the invention that inflammatory processes and diseases are beneficially influenced directly or indirectly by interaction, and an alleviation of disease symptoms and improvement in the progress of the disease occur. Interactions with reactive mediators of inflammations such as, for example, hypochlorous acid (hypochlorite) may play a particular role in this. However, this also means that the risk of inflammatory disorders, especially of the gastro-intestinal tract, can be reduced. Typical inflammatory disorders of the gastrointestinal tract which should be mentioned are, for example, intestinal inflammations (enteritis), gastroenteritis, nonspecific irritable bowel syndrome and the chronic inflammatory bowel disorders ulcerative colitis and Crohn's disease.

Chemopreventive means for the purpose of the invention that processes within tumorigenesis/carcinogenesis can be beneficially influenced directly or indirectly by interaction, and manifestation of the disease is prevented or the risk of becoming ill is reduced. Chemopreventive also means protection from genotoxic and mutagenic damage, especially DNA damage. Direct interactions with DNA-damaging agents and mutagens, but also procarcinogens requiring metabolic activation, may play a particular part in this. A further meaning thereof is that the growth of cancer cells can be inhibited (antiproliferative effect). Typical cancers of the gastrointestinal tract which should be mentioned include stomach cancer and colon cancer.

Carob products for the purposes of the invention are the natural carob products (Ceratonia siliqua) itself or secondary products obtained by means of technological processes such as, for example, mechanical size reduction, separation, grading, thermal treatment, extraction, extrusion, pressure treatment etc. The pulp of the carob fruit after removal of the seeds accounts for between 63 and 92% by weight of the whole fruit, depending on the species. The chemical composition of carob varies over a relatively wide range owing to its biological origin (depending inter alia on the variety, the climate, the soil etc.). The main components of carob are water-soluble sugars and may normally be present in the range from 30 to 60% by weight, preferably from 40 to 50% by weight.

The carob products employed in the compositions of the invention include all products produced by mechanical and physical size reduction, physicochemical processes (heating, extraction etc.) and/or other technological treatment and process steps. Particularly suitable carob products are carob fibers, preferably produced by the process disclosed in EP-A-0 616 780, which is incorporated herein by reference. The carob fibers obtained in this way are substantially insoluble in water, i.e. they still contain a maximum of 1 to 30% by weight, preferably 10 to 15% by weight, of water-soluble constituents.

The compositions of the invention may, besides the carob product, in particular the carob fiber, also comprise other components appropriate for the type of application. Besides direct consumption of carob products or direct addition by the consumer to food products and beverages of general consumption, they can also be added during the industrial manufacture of food products. Suitable in this case for the carob products, in particular the carob fiber, are conventional food product applications such as, for example, bakery products, cereals, snack bars or fruit bars or drinks powders. Use in dietary supplement- or medicament-typical form is also possible, such as, for example, tablets, coated tablets, capsules, sachets or granules. The anti-inflammatory and chemopreventive compositions in dietary supplement- or medicament-typical form may comprise conventional additives such as solvents, fillers, carriers such as methylcellulose, sweetening carbohydrates and other sweeteners, flavors, antioxidants, colors etc.

The compositions of the invention can also be employed directly in combination with the active pharmaceutical ingredients to be used for therapy. The best procedure for producing these combination products is to mix the required amounts of carob product, in particular carob fiber, and active ingredient, to spray dry, to remove solvent, to agglomerate and/or to instantize. It is furthermore possible to use all production processes customary in food technology, but also pharmaceutical technology, such as, for example, mixing, emulsifying, suspending, dispersing, and compressing, kneading or sugar-coating.

The carob products, especially carob fiber, are present in the compositions of the invention in concentrations which have a beneficial effect on the risk factors for the corresponding diseases and thus reduce the risk of inflammations or cancer, especially of the digestive tract, but also have a beneficial effect on and diminish the symptoms or molecular processes during the course of the diseases, and thus assist the process of cure. The daily dose of carob products can be, depending on the pathological state, the particular disease conditions, but also individual need, in the region of 0.1-50 g, normally 3-10 g. The additives which are present where appropriate in dietary supplement- or medicament-typical applications can expediently be added in concentrations of 1-90% by weight, in particular of 10-60% by weight (based on the composition).

The compositions of the invention are expediently employed in a suitable preparation which is adapted to the ratios of amounts with the best effect. Food product applications are, in particular, suitable for the carob products, in particular the carob fiber. Every food product into which the carob products, in particular the carob fiber, can be incorporated is suitable in principle, with limits emerging from the properties of the carob component and from the intended purpose of use. Accordingly, cereals-based food products are particularly suitable, such as bakery products, cereals, snack bars and fruit bars, desserts, sweets, special dietary preparations such as drinks and, in particular, drinks powders based on milk, fruit concentrates or powders, carbohydrates or sugar alcohols. The carob products, especially the carob fiber, can furthermore be consumed directly as foods, be mixed with meals prepared at home or be administered in dietary supplements. Conventional dosage forms for dietary supplements include tablets, capsules, powder preparation for dissolving or else granules.

If the compositions of the invention are administered directly to support a drug therapy, this can take place in separate form, but also within one product. Examples suitable for the latter variant are preparations in the form of powders or tablets for dissolving, but also chewable tablets. These preparations may additionally comprise further ingredients to improve the dissolving, such as soluble carriers, disintegrants, and generally colors, sweeteners such as sucrose, glucose, fructose and other carbohydrates, sugar alcohols such as, for example, sorbitol, xylitol, maltitol and isomalt, or sweetening agents such as, for example, acesulfame K, cyclamate, saccharin, sucralose or aspartame and, in particular, flavorings to improve acceptance.

The carob products, in particular carob fiber, surprisingly show that they are able to inactivate mediators of inflammation, such as, for example, hypochlorite (hypochlorous acid), which play an important part in inflammatory disorders, very effectively, and thus counteract their adverse, harmful effects. It is thus possible for various inflammatory factors to be neutralized effectively even prior to a potential inflammatory disorder of the digestive tract, which is associated with a marked reduction in the risk of inflammation, but also an alleviation of the symptoms of the disease.

It has additionally been found, surprisingly, that the carob products, especially carob fiber, likewise have chemopreventive properties. This is all the more surprising for the example of carob fiber because it is substantially insoluble and thus many readily soluble and bioavailable components are removed by aqueous extraction. It was possible nevertheless to show that carob fibers are able to protect intestinal cells efficiently from DNA damage, and thus the risk of possible mutation and initiation of the carcinogenesis process is reduced. It has furthermore been found, surprisingly, that extracts of carob products, especially carob fiber, obtained under physiologically relevant conditions are able to inhibit the proliferation of bowel cancer cells and thus reduce the risk of the occurrence of colon carcinoma.

The compositions of the invention thus allow various risk factors and mediators of inflammatory processes, but also of tumorigenesis in the gastrointestinal tract, to be reduced, which eventually on the one hand considerably reduces the general risk, and on the other hand can considerably alleviate the symptoms of the disease. Compared with a wide-ranging change in diet, the effects of the risk reduction through the compositions of the invention can be achieved in a considerably simpler manner (for example dietary supplements or bars with the composition of the invention), which represents a considerable improvement over the current state of the art (inter alia wide-ranging change in diet). The compositions of the invention are furthermore able to provide sensible support for therapeutic measures, which leads to desirable improvements in the progress of the disease. It is thus possible to reduce markedly the duration and the dose level of the medication. It is thus possible to reduce drastically the side effects associated with the medication, which represents a considerable advance in the drug therapy of inflammatory disorders of the gastrointestinal tract.

The invention is illustrated by means of examples below:

### EXAMPLE 1

Reactivity Toward the Mediator of Inflammation Hypochlorous Acid (HOCl)

Cytotoxic hypochlorous acid is a specific product of phagocytosis-competent neutrophilic granulocytes. It is used to kill phagocytosed bacteria and viruses inside these leukocytes. Hypochlorous acid is, however, secreted through the leukocyte membrane into the extracellular medium, i.e. into the surrounding tissue, and there proves to be responsible for unwanted reactions like those observed at foci of inflammation (pain, reddening, swelling).

A possible procedure for detecting the reactivity toward HOCI is as follows. HOCI liberates ethene specifically from ACC (1-aminocyclopropane-1-carboxylic acid), and the ethene is determined by gas chromatography. The following mixture is chosen to examine the interaction of the test substance in this model system:

| Reagents | Concentration in the mixture | |
|---|---|---|
| Phosphate buffer, pH 5.7 | 0.1 | M |
| Carob fibers | 0.005 to 1.0 | mg/2 ml |
| (Caromax ™*), Nutrinova) | | |
| HOCl | 25 | μM |
| ACC | 1 | mM |
| dd H₂O ad 2 ml | | |

*)prepared by the process of EP-A-0 616 780

The hypochlorous acid is allowed to interact with the bulking agents for 30 seconds and only then is the indicator molecule ACC added. The volume-calibrated reaction vessels which are closed gas-tight are then incubated at 37° C. for 30 minutes, and the ethene liberation is measured by GC. 5-aminosalicylate is employed in concentrations of 0.5-100 μM as positive control. The results of this test are shown in table 1.

TABLE 1
Reactivity of carob fibers toward HOCl

| | | Amount of ethene | | Amount of ethene |
|---|---|---|---|---|
| 5-Amino- | | liberated from ACC | | liberated from ACC |
| salicylate | | in % | Carob fiber | in % |
| 0.5 | μM | 100 | 0.005 mg/2 ml | 93 |
| 1 | μM | 92 | 0.01 mg/2 ml | 81 |
| 5 | μM | 52 | 0.05 mg/2 ml | 57 |
| 10 | μM | 13 | 0.1 mg/2 ml | 37 |
| 50 | μM | 5 | 0.5 mg/2 ml | 11 |
| 100 | μM | 3 | 1 mg/2 ml | 10 |

The carob fibers show in this test system an exceptionally high reactivity toward HOCl. Compared with the anti-inflammatory agent 5-aminosalicylate, the carob product shows a very similar reactivity and is able efficiently to inactivate this mediator of inflammation and thus alleviate the symptoms of inflammatory disorders.

### EXAMPLE 2

Protection of Intestinal Cells from Genotoxic Agents

This example shows the protective effect of extracts of carob fiber on colon cells in culture. The procedure for the method is as follows:

The human colon adenocarcinoma cell line Caco-2 was cultivated in Dulbecco's MEM/nut mix F-12 Ham medium (Gibco) with 20% FCS (Gibco) and 1% penicillin/streptomycin (Gibco). The cells were seeded in Petri dishes (ø 35 mm) 24 h before substance incubation. After incubation with the physiological extract from carob fiber (stock solution: 10% w/v carob fiber, Caromax™, Nutrinova, in cell culture medium, working solutions: 0.001-1%), the cells were washed with PBS buffer solution (phosphate buffered salt solution) and subsequently treated with the DNA-damaging agent menadione (2 μM) and trypsinized/centrifuged.

This was followed by testing for the preventive activity of the carob fiber extracts.

The modulation of DNA damage by the carob fiber extracts compared with the untreated control was checked by single-cell gel electrophoresis (comet assay). The extent of DNA damage was quantified from the proportion of DNA in the comet tail (% tail intensity, TI %). The independent baseline damage was always also measured in control tests. The results are summarized in table 2:

TABLE 2
Protective effect of carob extract (CE) on Caco-2 cells

| Substance | TI % |
|---|---|
| Control | 2.6 |
| menadione 2 μM | 13.6 |
| CE 0.001% | 13.9 |
| CE 0.01% | 12.0 |
| CE 0.03% | 7.1 |
| CE 0.1% | 3.2 |
| CE 0.3% | 5.1 |
| CE 1% | 7.6 |

The DNA damage caused by menadione, expressed by a TI of 13.6, can be reduced by carob fiber extracts as far as the control level (no induced DNA damage), depending on the concentration. The tested carob product thus shows an extremely pronounced activity in relation to DNA damage in intestinal cells. This protective effect reduces DNA damage initiating the cancer process and thus reduces the risk of cancer.

### EXAMPLE 3

Antiproliferative Effect of Carob Products on Bowel Cancer Cells

The antiproliferative effect of extracts of carob fiber on HT29 cells kept in culture is shown in this example. The procedure for the method is as follows:

The human colon cancer cell line HT29 was cultivated in Dulbecco's modified Eagle medium (Gibco) with 10% FCS (Gibco) and 5% penicillin/streptomycin (Gibco). The cells were seeded in 96-well plates to determine the proliferation. Incubation with the carob fiber extract (stock solution: 10% w/v carob fiber, Caromax™, Nutrinova, in cell culture medium, working solutions: 0.05-2%) took place after 24 h. After an incubation time of 72 h, the DNA content is determined as a measure of the proliferation using DAPI (4′,6-diamidino-2-phenylindole dihydrochloride) staining.

The results of the proliferation test are summarized in table 3:

TABLE 3
Antiproliferative effect of carob extract (CE) on HT29 cells

| CE concentration in % | Proliferation in % |
|---|---|
| control | 100 |
| 0.05 | 97 |
| 0.1 | 93 |
| 0.2 | 85 |
| 0.3 | 69 |
| 0.4 | 55 |
| 0.6 | 32 |
| 1 | 15 |
| 2 | 9 |

Carob fiber extracts significantly modulate the growth of human colon cancer cell lines. The IC50 (value at which 50% of the cells no longer grow) is 0.42% by weight. Carob products thus have a significant effect on the process of colon carcinogenesis.

### EXAMPLE 4

Chewable Tablet

The chewable tablets were produced in accordance with the following formula:

| | | |
|---|---|---|
| Carob fiber (Caromax ™, Nutrinova) | 2 | g |
| Sorbitol (Roquette) | 1.4 | g |
| Magnesium stearate (Merck) | 15 | mg |
| Acesulfame K (Sunett ®, Nutrinova) | 12 | mg |
| Aspartame (Holland Sweetener Corp.) | 12 | mg |

| | |
|---|---|
| Chocolate flavor (Danisco) | q.s. |

The ingredients of the chewable tablets are mixed dry and compressed in a conventional way.

### EXAMPLE 5

Fruit Bar

The fruit bars were produced in accordance with the following formula:

| | |
|---|---|
| Orange fruit preparation (Zentis) | 6 g |
| Marzipan | 5 g |
| Candied orange peel | 5 g |
| Carob fiber (Caromax ™, Nutrinova) | 5 g |
| Figs | 3 g |
| Sultanas | 2 g |
| Apples | 2 g |

All the ingredients are mixed together and finely comminuted. The composition is then pressed in a mold, and the tops and bottoms of the resulting bar cores are covered with sheets of rice paper.

1. A dietary supplement which comprises a carob product.

2. A dietary supplement according to claim 1 which is an anti-inflammatory dietary supplement.

3. A dietary supplement according to claim 1 which is a chemopreventive dietary supplement.

4. The dietary supplement as claimed in claim 1, which comprises carob fruit pulp from which the seeds have been removed.

5. The dietary supplement as claimed in claim 1, which comprises carob fiber.

6. The dietary supplement as claimed in claim 1, which consists of carob fiber.

7. The dietary supplement as claimed in claim 5, wherein the carob fibers still comprise from 1 to 30% by weight of water-soluble constituents.

8. The dietary supplement as claimed in claim 1, which comprises an additional active ingredient.

9. The dietary supplement as claimed in claim 1 in the form of a food product.

10. The dietary supplement as claimed in claim 1 in the form of a beverage.

11. The dietary supplement as claimed in claim 1 in the form of a food additive.

12. The dietary supplement as claimed in claim 1 in the form of an animal feed.

13. The dietary supplement as claimed in claim 8 in the form of a medicament.

14. The dietary supplement as claimed in claim 8 in the form of a veterinary medicament.

15. A process for producing a dietary supplement having anti-inflammatory activity, which comprises converting a carob product into a dietary supplement, having anti-inflammatory activity.

16. A process for producing a dietary supplement having chemopreventive activity, which comprises converting a carob product into a dietary supplement, having chemopreventive activity.
